(19) United States
(12) Patent Application Publication
(10) Pub. No.: US 2025/0054378 A1
(43) Pub. Date: Feb. 13, 2025

(54) METHOD AND APPARATUS FOR PREVENTING ELOPEMENT

(71) Applicant: INVISALERT SOLUTIONS, INC., West Chester, PA (US)

(72) Inventor: Peter A. NAGY, West Chester, PA (US)

(21) Appl. No.: 18/705,441

(22) PCT Filed: Oct. 26, 2022
(86) PCT No.: PCT/US2022/078716

§ 371 Date: Apr. 26, 2024

Related U.S. Application Data
(60) Provisional application No. 63/272,150, filed on Oct. 26, 2021.

(51) Int. Cl. G08B 21/02; G16H 40/67; H04W 4/029

(52) U.S. Cl. CPC G08B 21/02; G16H 40/67; H04W 4/029

(57) ABSTRACT

Methods and apparatuses (e.g., tracking system, tracking devices, etc.) for tracking a subject to prevent elopement. The methods and apparatuses described herein may be used to prevent elopement from health care environments. The methods and apparatuses may be specifically configured to provide energy and cost efficient mechanisms to prevent elopement and to track subjects. For example, these methods and apparatuses may monitor, using a wearable tracking device, low-energy sensor data and determining whether the sensor data exceeds a threshold. If the sensor data exceeds a threshold, then the tracking device can turn on scanning for a wireless signals associated with a known location, and/or activate a location-determining subsystem of the tracking device and transmit location information and/or an alert to a remote processor.

### CROSS REFERENCE TO RELATED APPLICATIONS

This patent application claims priority to U.S. provisional patent application No. 63/272,150, filed on Oct. 26, 2021, titled “METHOD AND APPARATUS FOR PREVENTING ELOPEMENT” and herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Described herein are apparatuses (e.g., devices, systems etc.) and methods for detecting elopement of a subject and/or tracking the subject. In particular, the apparatuses described herein may detect elopement, track one or more subjects, and warn supervisory personnel when the subject(s) have left a prescribed boundary, such as a healthcare facility, without authorization.

An elopement may refer to a subject leaving a prescribed environment, such as a healthcare facility (e.g., emergency room, hospital, psychiatric ward, residential facility, skilled nursing facility, or the like), without authorization. In such circumstances the subject may be a danger to themselves or to others, and/or may be under a legal or medical statuses. Elopement can have serious consequences for the subject as well as the facility and caregivers. Subjects can experience death or injury during an elopement. Furthermore, healthcare facilities and caregivers often have a legal liability for eloped subjects which can result in substantial negligence claims. Elopements can also lead to punitive consequences by licensing, regulatory, and/or insurance bodies. In some settings, elopements can also result in a loss of revenue and/or lower reimbursement rates.

Healthcare facilities can be busy and chaotic, especially in an emergency room (ER). Subjects can elope without notice and often there is no alerting in place to let staff know that the subject has eloped. RTLS (real-time location systems) are sometimes used in hospitals to provide location of subjects or assets. Generally, these systems are expensive to buy, install and maintain. Many RTLS systems generally do not provide any tracking ability beyond a building perimeter and typically do not have any tamper-resistance with respect to a subject worn tracking device.

Thus, there exists a need for a subject tracking device that can work in a variety of locations.

### SUMMARY OF THE DISCLOSURE

The methods and apparatuses (e.g., systems, devices, etc.) described herein may be useful for identifying elopement, e.g., identifying when a subject, such as but not limited to a patient, leaves a prescribed area, such as a room, ward, floor, building, campus, etc. These methods and apparatuses may also be used to track one or more subjects that have left a prescribed region. In particular, these methods and apparatuses may provide accurate methods that are both energy efficient and highly accurate and precise. Normally, it may be difficult to provide both energy efficiency and precision in determining elopement and/or tracking; precision in tracking typically requires the use of power intensive systems. As a result other system may provide tracking only relatively infrequently, and/or low resolution.

The methods and apparatuses described herein may include tracking devices that may allow monitoring in a low-cost and energy efficient manner by performing energy-intensive steps such as wirelessly confirming proximity to one or more regions and/or tracking location only when triggered by one or more subject or environmental triggers that are correlated with a likelihood of elopement. The method described herein (and apparatuses for performing them) may monitor, by the tracking device, sensor data, determining whether the sensor data exceeds a threshold, scanning for wireless signals in response to determining that the sensor data exceeds a threshold, matching the scanned wireless signals with wireless signals associated with known locations, and enabling location determining operations in response to matching the scanned wireless signals with wireless signals associated with known locations.

In some examples, the sensor data may include inertial sensor data, environmental sensor data, or a combination thereof. Furthermore, wherein the inertial sensor data may include accelerometer data, footfall data, falling data, or a combination thereof. In some cases, the environmental sensor data may include ambient temperature, ambient light data, sound, pressure (e.g., barometric pressure), or a combination thereof.

In some examples, the scanned wireless signals may include periodic signals transmitted by other wireless devices. In some other examples, the periodic signals may include Wi-Fi beacon signals.

In some examples, the scanned wireless signals may be a radio-frequency signal. The wireless signal may include at least one of a Wi-Fi signal, an ultrawideband (UWB) signal, a Bluetooth signal, or a combination thereof. For example, the wireless signal may be used to look for presence of signal, and in any of the apparatuses and methods described herein the wireless signal (e.g., UWB) may also be used to determine a distance from source, e.g., a using time of flight determination. In some other examples, the wireless signals associated with known locations may be stored in a memory of the tracking device. In still other examples, the method may include determining a location of the tracking device based on the location determining operations and notifying monitoring personnel in response to determining the location of the tracking device. In some cases, the notification to the monitoring personnel may include the determined location of the tracking device.

Also described herein are tracking devices. A tracking device may include a controller configured to monitor sensor data and determine whether the sensor data exceeds a threshold. The tracking device may also include a wireless transceiver configured to scan for wireless signals in response to a determination that the sensor data exceeds a threshold and a location determination unit. The location determination unit may be configured to perform location determining operations. The controller may further be configured to match the scanned wireless signals with wireless signals associated with known locations and enable location determining operations in response to a match between scanned wireless signals and wireless signals associated with known locations.

In some examples, the sensor data may include inertial sensor data, environmental sensor data, or a combination thereof. In some cases, the inertial sensor data may include accelerometer data, footfall data, falling data, or a combination thereof. In some other cases, the environmental sensor data includes ambient temperature, ambient light data, pressure, sound, etc. or a combination thereof.

In some examples, the scanned wireless signals may include periodic signals transmitted by other wireless devices. The periodic signals may include Wi-Fi beacon signals. In some other examples, the scanned wireless signals include at least one of Wi-Fi signals, Bluetooth signals, or a combination thereof.

In some examples, the location determination module may be further configured to determine a location of the tracking device and the wireless transceiver may be further configured to transmit a notification message to monitoring personnel in response to a determination of the location of the tracking device. In some cases, the notification message may include the location of the tracking device.

The methods and apparatuses described herein can be implemented as a method for tracking a subject. The method may include scanning, by one or more wireless access points, for identification signals associated with individual subjects, matching the scanned identification signals with predetermined identification signals, wherein the predetermined identification signals are associated with individuals expected to be within wireless range of the one or more wireless access points, determining that at least one of the predetermined identification signals fails to match with the scanned identification signals, and notifying monitoring personnel in response to determining that at least one of predetermined identification signals fails to match with the scanned identification signals.

In some examples, the identification signals may be transmitted by a wireless transmitter worn by individual subjects. In some other examples, the identification signals may include Wi-Fi signals, Bluetooth signals, Zigbee signals, or a combination thereof. In still other examples, the one or more wireless access points may be coupled to a monitoring server configured to match scanned identification signals to the predetermined identification signals.

In some examples, the notifying may include transmitting alert messages to the monitoring personnel. In some examples, the alert message may be transmitted to at least one of a desktop computer, laptop computer, personal digital assistant, tablet computer, and smart phone. In some other examples, the alert message may be transmitted by a server coupled to the one or more wireless access points.

Also described herein are subject tracking systems. The system may include one or more wireless access points configured to scan for identification signals associated with individual subjects, and a monitoring server configured to match the scanned identification signals with predetermined identification signals, wherein the predetermined identification signals are associated with individuals expected to be within wireless range of the one or more wireless access points, determine that at least one of the predetermined identification signals fails to match with the scanned identification signals, and notify monitoring personnel in response to a determination that at least one of predetermined identification signals fails to match with the scanned identification signals.

In some examples, the identification signals may be transmitted by a wireless transmitter worn by individual subjects. In some other examples, the identification signals may include Wi-Fi signals, Bluetooth signals, Zigbee signals, or a combination thereof. In still other examples, the one or more wireless access points may be coupled to a monitoring server configured to match scanned identification signals to the predetermined identification signals. In some examples, the monitoring server may be further configured to transmit alert messages to the monitoring personnel.

For example, described herein are methods for tracking a subject that preserves battery life in a tracking device worn by a subject. Any of these methods may include: monitoring sensor data in the tracking device; determining whether the sensor data exceeds a threshold; turning on scanning for a wireless signal when the sensor data exceeds the threshold; and enabling location-determining operations when the scanned wireless signal does not match a wireless signal associated with a known location. The sensor data may be subject movement sensor data, environmental sensor data, or a combination thereof. The subject movement sensor data may be accelerometer data, footfall data, falling data, or a combination thereof. For example, the environmental sensor data may be ambient temperature, ambient light data, pressure, sound, etc. or a combination thereof.

In any of these methods and apparatuses, the wireless signal may be a signal transmitted by a wireless access point. The wireless signals may be a Wi-Fi beacon signal. In some examples, the wireless signal is a Wi-Fi signals, a Bluetooth signals, or a combination thereof. The apparatus may be configured to recognize the local wireless access point by name and/or address. The wireless signals associated with the known location may be stored in a memory of the tracking device, e.g., during an initialization phase.

Any of these methods may include determining a location of the tracking device based on the location determining operations; and notifying monitoring personnel in response to determining the location of the tracking device. For example, notifying monitoring personnel may include transmitting a determined location of the tracking device to the monitoring personnel.

Any of the location determining operations may be performed every minute or more frequently (e.g., at a frequency of 0.02 Hz or greater). Enabling the location-determining operations may include using one or more of: global positioning system (GPS) signals indicating the location of the tracking device, public WiFi signals, and/or barometric pressure. Any of these methods and apparatuses may be configured to use public WiFi addresses to determine location. For example, using databases including maps of WiFi MAC addresses correlated to GPS locations. Such databases are created and maintained by third parties (e.g., Google) that capture this information off of cell phones. Any of these methods and apparatuses may also and/or alternatively use pressure, e.g., barometric pressure data (altitude) to determine location of the subject-worn apparatus, as described herein. Barometric pressure may be converted into altitude and may be used in combination with a databases that includes altitude data correlated with latitude/longitude coordinates. In any of these methods and apparatuses one or more (e.g., any combination of these) location determining components may be used. In some examples, these components may be aggregated (e.g., GPS, WiFi, altitude, etc.) to enhance location accuracy. Alternatively, any of these components can be used individually.

Monitoring the sensor data in the tracking device may include monitoring a running average of the sensor data. For example, sensor data may be monitored continuously or periodically (e.g., at a first sampling rate). The method or apparatus may determine a running window of time over which the sensor data may be examined (e.g., averaged, smoothed, etc.). In some examples the monitoring may monitor the instantaneous value from the one or more sensors; in other examples the monitoring may monitor a running window and may, for example, look at the average within the running window. The window may be over a predefined time period of, e.g., one second, one 2 seconds, 4 seconds, 5 seconds, 30 seconds, 1 minute, 5 minutes, 10 minutes, 15 minutes, 20 minutes, 30 minutes, 35 minutes, 45 minutes, 50 minutes, 60 minutes, etc.

Any of these methods (or apparatuses configured to perform them) may include turning off scanning for the wireless signal if the wireless signal matches the wireless signal associated with the known location. The apparatus (or system) may reset monitoring of the sensed data (e.g., locomotion) or a running window (e.g., running average, etc.) when monitoring.

For example, described herein are methods for tracking a subject that preserves battery life of a tracking device worn by the subject, the method comprising: monitoring subject locomotion using the tracking device, wherein the tracking device includes one or more motion sensors; determining whether the subject locomotion exceeds a distance threshold and/or exceeds a rate threshold indicating running; turning on scanning for a wireless signal by the tracking device if the subject locomotion exceeds the distance threshold and/or exceeds the rate threshold indicating running; and enabling location-determining operations in the tracking device if the tracking device identifies a wireless signal that matches a known location, wherein the location-determining operations include turning on location services and relaying position from the tracking device to a remote processor at a rate of 0.02 Hz or greater.

As mentioned, also described herein are tracking devices configured to perform any of these methods. For example, a tracking device may include: one or more of: a motion sensor and an environment sensor; a wireless transceiver configured to scan for a wireless signal, wherein the wireless transceiver is configured to be unpowered until activated; a location determination unit configured to perform location determining operations at a frequency of 0.02 Hz or greater in a powered state, wherein the location determining unit is unpowered until activated; and a controller comprising one or more processors, wherein the controller is configured to: monitor sensor data from the one or more of the motion sensor and environmental sensor; determine whether the sensor data exceeds a threshold; activate the wireless transceiver to scan for the wireless signal when the sensor data exceeds the threshold; and activate the location-determining unit when the scanned wireless signal does not match a wireless signal associated with a known location to determine a location of the tracking device, and transmit the location to a remote processor. The motion sensor and the environment sensor may include an inertial sensor, an environmental sensor, or a combination thereof. The motion sensor and the environment sensor may include an accelerometer. The motion sensor and the environment sensor may include an ambient temperature sensor, an ambient light sensor, pressure sensor, acoustic sensor, etc. or a combination thereof. The wireless transceiver may be configured to detect a wireless access point. The wireless transceiver may be configured to detect periodic signals from a Wi-Fi beacon. The wireless transceiver may be configured to detect at least one of Wi-Fi signals, Bluetooth signals, or a combination thereof. The wireless transceiver may be further configured to transmit a notification message to a monitoring personnel including the location of the tracking device.

The location determination unit may be configured to determine the location of the tracking device by global positioning system (GPS) signals, as mentioned above.

For example, a tracking device may include: a motion sensor; a wireless transceiver configured to scan for a wireless signal, wherein the wireless transceiver is configured to be unpowered until activated; a location determination unit configured to perform location determining operations at a frequency of 0.02 Hz or greater in a powered state, wherein the location determining unit is unpowered until activated; and a controller comprising one or more processors, wherein the controller is configured to: monitor a subject's locomotion using the motion sensor; determine whether the subject's locomotion exceeds a distance threshold and/or exceeds a rate threshold indicating running; activate the wireless transceiver to scan for the wireless signal when the subject's locomotion exceeds the distance threshold and/or exceeds the rate threshold indicating running; and activate the location-determining unit when the scanned wireless signal does not match a wireless signal associated with a known location to determine location information, and transmit the location information to a remote processor at a rate of 0.02 Hz or greater.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 illustrates one example of a block diagram of a tracking device.

FIG. 2A is a diagram depicting an example deployment of a subject elopement tracking system.

FIG. 2B is a diagram showing an example deployment of an elopement tracking and prevention system as described herein.

FIG. 2C schematically illustrates an example deployment of an elopement tracking and prevention system as described herein.

FIGS. 3A-3C illustrate examples of different use cases for the elopement tracking and prevention systems described herein. FIG. 3A shows an example of a facility having both buildings and outside space, in which the boundary of the facility overlaps with the building. FIG. 3B shows an example of a facility having multiple buildings within an outdoor space. FIG. 3C shows an example of an all-indoor space (e.g., hospital floor or region).

FIG. 4A is an example of a flowchart depicting a method for detecting elopement and/or tracking a subject wearing a tracking device.

FIG. 4B is an example of a flowchart depicting a method for detecting elopement and/or tracking a subject wearing a tracking device including a motion sensor.

FIG. 5A is one example of a flowchart depicting a method for detecting elopement and/or tracking one or more subjects.

FIG. 5B is an example of a flowchart depicting a method for detecting elopement and/or tracking one or more subjects.

### DETAILED DESCRIPTION

Described herein are methods and apparatuses for detecting patient elopement and/or for tracking patients that have eloped. Systems that track a subject (e.g., patient, child, pet, dementia patient, etc.) wearing a wristband, collar or tag may interact with wireless technology in and out of a dedicated home-base unit. Typically, these systems may have the wristband checking for the presence of a home-based signal such as a home Wi-Fi or Bluetooth signal such as from the home or a phone. The frequency of these home-base checks needs to be fast enough such that the tracked subject couldn't travel too far before an alert is triggered. In the absence of the home-based signal, the wristband may turn-on a locating service and report the subject's location via some type of back-haul technology (e.g., cellular, LoRa) to a central processing unit (CPU). The CPU is typically configured to send alerts and tracking information to a designated recipient's phone or desktop. The locating service might be GPS or a homing-signal. GPS can fix position using satellite information. A homing-signal might rely on a wireless antenna array to fix the position of the subject. Wireless antenna array technology could be, among other things, cell towers, Wi-Fi, LoRa, Zigbee, etc. These array technologies typically use some type of multilateration to establish a position fix based on the wristband's homing signal.

However, such systems (which may be referred to as indoor-to-outdoor alerting and tracking systems) may unfortunately have a short battery life. Many of the location and back-haul technologies are inherently power hungry, (e.g., GPS and cellular). However, another aspect of these systems that may also have a large power requirement is the indoor home-base signal monitoring. Even if the wristband only checks (e.g., in a receive mode) for the home-base signal every 5 minutes, it can still be much more power consumptive than systems that transmit alone (transmit mode). As a result, some systems only check for the presence of the home-base signal relatively infrequently, (i.e., 5 min or longer intervals), even though these long interval checks may allow the tracked subject to travel a lot of ground before being located.

The methods and apparatuses described herein may provide accurate and power-efficient ways to detect elopement and/or track a subject. In general these methods and the apparatuses (devices, systems, etc.) configured to perform them may use one or more low-power sensors for continuous or periodic (including relatively high frequency) monitoring of a characteristic of the subject and/or an environmental characteristic in order to trigger detection of proximity to a beacon and/or a wireless signal that is correlated with an expected location for the patient in order to determine elopement; once elopement is detected by failure to detect a wireless signal (e.g., wireless network), the method or apparatus may determine a location for the eloping subject. The location may be detected at a relatively high frequency, even though it requires a lot of energy.

In general, the characteristic of the subject may be a physical characteristic of the patient, such as movement (e.g., walking/running gait, distance of continuous travel/steps, etc.), heart rate, etc. The environmental characteristic may include detecting a property that typically differs from an internal/indoor location as compared to an external/outdoor location, such as the frequency or intensity of light, temperature, humidity, barometric pressure, etc. Either or both physical characteristics of the patient and/or environmental characteristics may be detected.

The apparatuses described herein may be configured to perform any of these methods and may generally be referred to as “tracking devices”. As used herein, a tracking device may refer to tracking in the broadest sense, including tracking as simply detecting elopement. In some examples, tracking may refer to more precise determination of position relative to a building, street or other landmark.

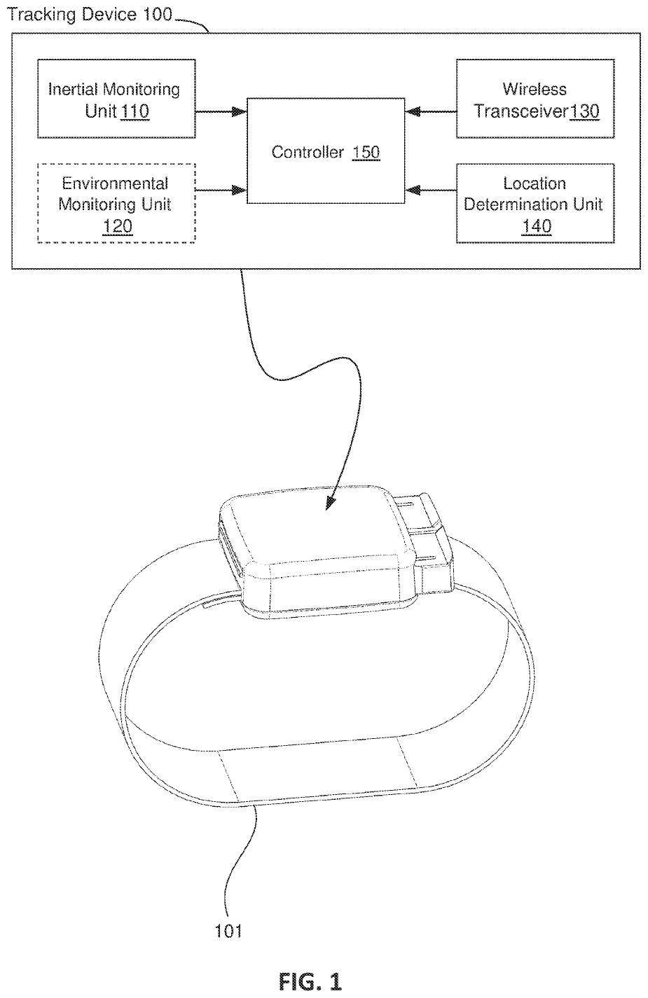

For example, FIG. 1 shows a block diagram of an example of a tracking device 100. The tracking device 100 may be used to track a location of a subject, and/or to determine if a subject elopement may be occurring. The tracking device 100 may include an inertial monitoring unit 110, an environment monitoring unit 120, a wireless transceiver 130, a location determining unit 140, and a controller 150. The inertial monitoring unit (IMU) 110, the environmental monitoring unit (EMU) 120, wireless transceiver 130, and location determining unit 140 may be communicatively coupled to the controller 150. Further, the tracking device 100 may include and be powered by a battery (not shown). In some variations, the tracking device 100 may be mounted within a housing 101 that may be conveniently worn by the subject, such as (in this example) worn about a subject's wrist. In some cases, the housing 101 may include a tamper-resistant clasp. Alternatively, the tracking device may be worn on a garment, or otherwise attached to the subject.

The tracking device 100 may operate in two or more operating modes. In a first operating mode, the IMU 110 of the tracking device 100 may monitor movements of the subject. The first mode may be relatively low power, as monitoring the IMU and/or EMU may require very little battery power. In some variations, the IMU 110 may include various accelerometers that may detect motion associated with footfalls (e.g., walking, jogging, running, etc.), falls (e.g., subject falling, stumbling, etc.), and the like. The IMU 110 may also include low-power state machines that can monitor IMU information constantly or periodically while consuming relatively low amounts of power. In a similar manner, the environmental monitoring unit (EMU) 120 may include one or more sensors for detecting environmental parameters, for example, temperature and light sensors that can detect changes near and around the tracking device 100. In some embodiments, the EMU 120 may include biometric sensors to track breathing, pulse rate and the like of the subject. For example, the EMU 120 may detect a temperature change (greater than a threshold) that would be indicative of the subject leaving a building (e.g., moving from a room temperature of about 78 degrees F. to a temperature higher or lower than room temperature). In some variations, the EMU 120 may detect a change in ambient lighting that may be associated with leaving an area. For example, the EMU 120 may detect a change from low light (during typical sleeping periods) to high light indicating that the subject is not sleeping and may be moving about. The tracking device may also include a clock; the clock information (time of day) may be used with the light information to determine outdoor/indoor status and therefore a likelihood of elopement.

In any of these apparatuses (e.g., systems, devices, etc.) described herein, the apparatus may include a sensor for sensing barometric pressure, which may be correlated to elevation and may be used in a topographic map. For example, if the area (e.g., facility) within which the patient is expected and/or permitted to roam and/or the surrounding region has a lot of topography/steep terrain, the barometric pressure may be used to determine elevation. For example, the barometric pressure may be used with a reference to atmospheric changes. Thus, in some examples in which the subject- (e.g., wrist-) worn device is configured to turn on “sniffing” (e.g., turn on a wireless transceiver for detecting/receiving a wireless signal) the one or more sensors that may be used to determine when to turn on and/or off may include a barometric pressure sensor. In some examples the apparatus may use topographic elevation to turn on/off a wireless receiver. In some examples the barometric pressure may be a unique point on a map or may otherwise help indicate the location (or leaving a location) and trigger turning on/off the wireless transceiver. Thus, in any of these apparatuses and methods the use of barometric pressure may enhance location determination and may provide another potentially unique data point.

In general, any of these examples the controller (including one or more processors) may use data from the IMU and/or EMU to determine if a patient is likely to have eloped (e.g., left the monitoring area). For example, the controller may determine, based on the number of continuous steps over time and/or the number of estimated “turns” if the subject has traveled in a particular direction longer than threshold that is set based on an approximate size of the monitoring area (e.g., ward, campus, building, etc.). Alternatively or additionally, the controller may determine, based on the gait (walking, running, etc.) or speed of travel (e.g., waling quickly, running), if the patient is likely to have eloped from the monitoring area. For example, the IMU data, which may be referred to as and may be processed as the subject's locomotion or locomotion data, may be compared to one or more thresholds (rages).

In some embodiments, the controller 150 may use dead reckoning techniques to determine if the tracking device 100 has left an area. Dead reckoning techniques may combine data from a variety of sensors, including sensors within the IMU 110 and the EMU 120. Dead reckoning techniques may determine steps, turns, and/or a direction of the subject.

Changes greater than a threshold may be determined by the IMU 110, the EMU 120, or the controller 150. Upon determination of a change greater than a threshold, the controller 150 can enable (in some cases power on) the wireless transceiver 130. The wireless transceiver 130 may include a wireless transmitter and a wireless receiver (not shown). The controller 150 may configure the wireless transceiver 130 to scan and/or receive Wi-Fi beacons from nearby wireless access points. In this manner, the wireless transceiver 130 may otherwise be in a powered-down or low power state, thereby conserving battery power of the tracking device 100.

In some cases, when a single threshold is exceeded, the controller 150 can enable the wireless transceiver 130 to scan for wireless signals. In some other cases, two or more thresholds need to be exceeded before the controller 150 enables the wireless transceiver 130 to scan for wireless signals. By combining multiple threshold events, the likelihood of a spurious enabling of the wireless transceiver 130 is reduced.

In general, the thresholds may be determined by a user, or may be automatically or semi-automatically set by the system, including by a user interface at the setup of the system. The system may be set up to establish parameters for each of one or more tracking devices and may include associating each tracking device with a subject (uniquely). The association between the tracking device and the subject/patient may be stored in a remote database that may be accessed by the system as described herein. The setup may associate the tracking device and/or subject with a local wireless network (e.g., access point, beacon, etc.). The setup may also include inputs from a user regrading the size, type and/or layout of the area that the subject is able to travel/remain in (e.g., size of the space, number of floors/elevation changes, number of rooms, hallways, buildings, if the space has indoor only or indoor/outdoor spaces, etc.). Thus, the system may determine one or more thresholds to help approximate elopement from the IMU/EMU data during an initiation or setup phase for one or more tracking devices. A user interface may be used to assist and simplify this process so that user input may be translated by the system into threshold values.

In operation, if the controller determines, based on one or more comparisons to IMU/EMU data to preset thresholds, that the subject may be eloping, the system may only then turn on the wireless transceiver for detecting (receiving) wireless signals. If the wireless transceiver 130 receives wireless signals from “known” wireless access points, then the controller 150 can determine that the location of the tracking device 100 is within a “known” area. That is, wireless signal characteristics may be stored within the tracking device 100 (e.g., stored in a memory coupled to the controller 150). Wireless signal characteristics may include various Wi-Fi beacon identifiers, SSID names, MAC address and the like. These wireless signal identifiers may be associated with wireless access points that are within a predetermined areas accessible by the subject. Therefore, receiving a wireless signal with characteristic that matches a stored wireless signal characteristic may confirm that the tracking device 100 is within a predetermined area.

In some embodiments, other wireless signals may be used to identify known areas. For example, Bluetooth signal identifiers can be stored within the tracking device 100 and used to determine the location of the tracking device 100. In a similar manner Zigbee signals, mobile phone tower signals, or any other wireless signals may be used to determine if the tracking device is within a predetermined area.

If the tracking device 100 is within a predetermined area, then the tracking device can return to monitoring the IMU 110 and the EMU 120. If the tracking device 100 is not within a predetermined area, then the tracking device 100 can determine its location through the location determining unit 140.

The location determination unit 140 may, for example, begin receiving and decoding navigation signals such as Global Positioning Signals (GPS), Global Navigation Satellite System (GLONASS) signals, BeiDou Navigation Satellite System signals (BEIDOUJ), or any other feasible navigation signals. In some variations, the location determination unit 140 may determine the location of the tracking device 100 using Angle of Arrival (AoA), Angle of Departure (AoD). Time of Flight (ToF), or any other feasible Wi-Fi or wireless signal based location determining procedure. In still other variations, the location determination unit 140 may use received signal strength indicators (RSSI) to determine the location of the tracking device 100.

In some embodiments, the location determination unit 140 may operate periodically, instead of continuously. More frequency operation may provide more frequent updates to the determined position of the subject. However, more frequent operation may also deplete battery power more quickly.

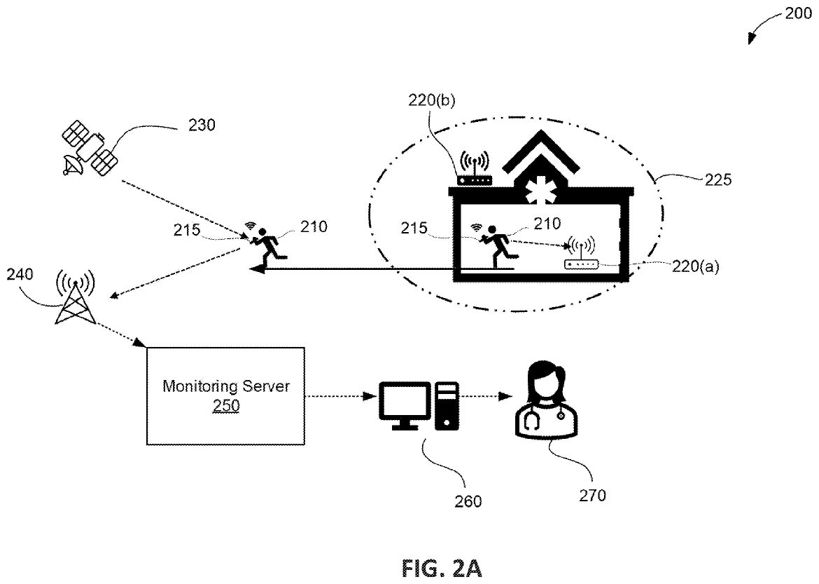

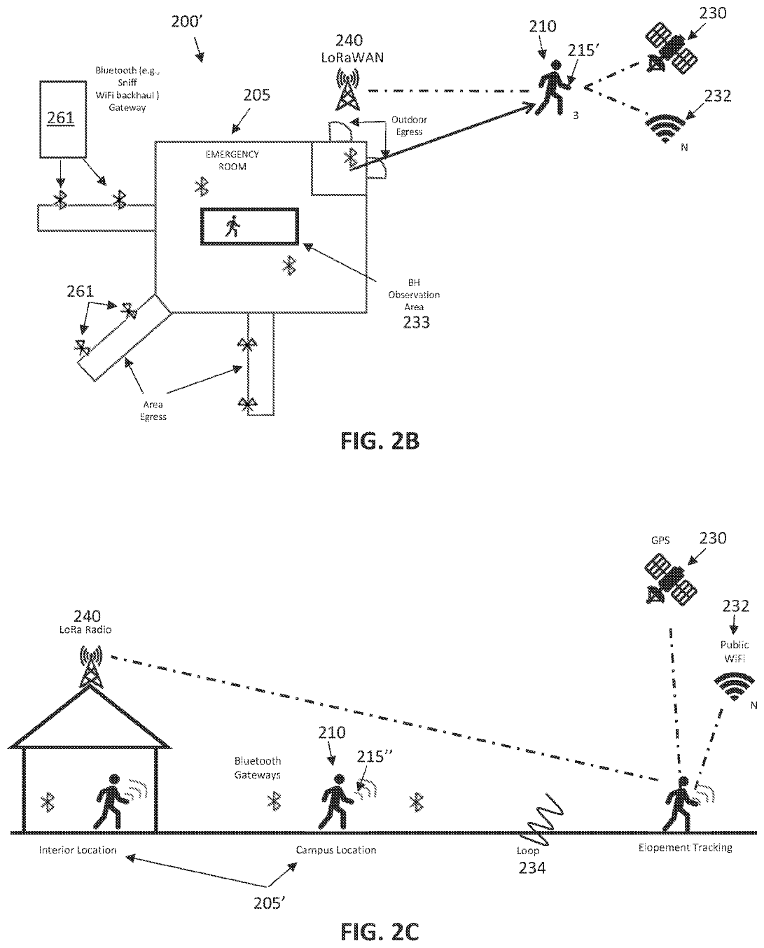

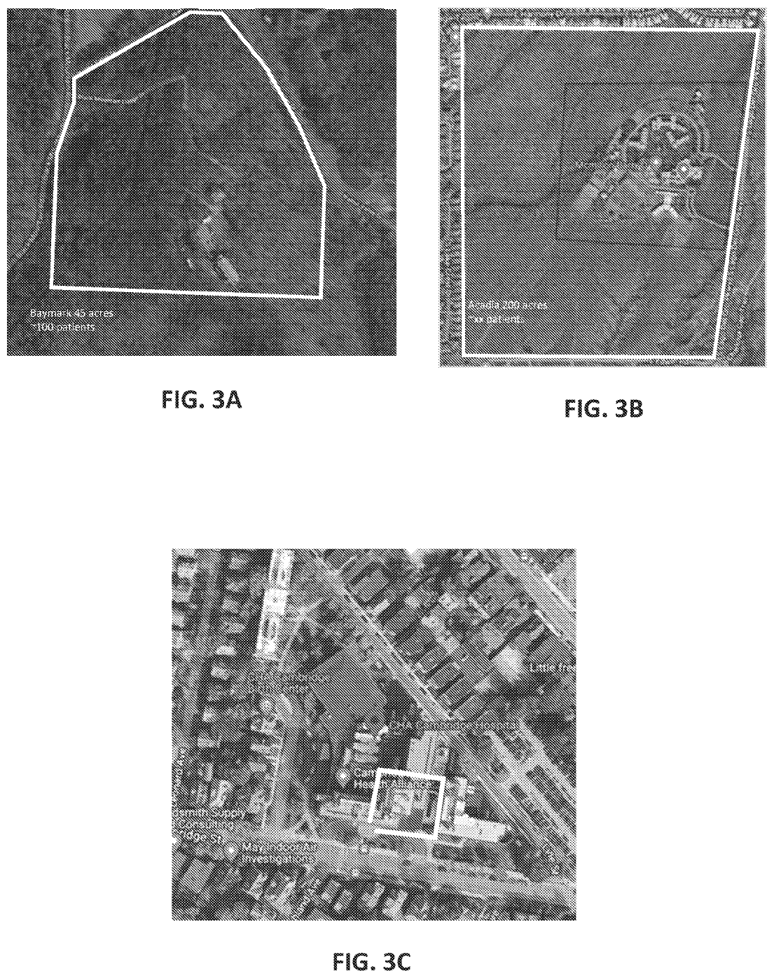

After the location is determined, the controller 150 may report its location to monitoring personnel by transmitting data through the wireless transceiver 130. In some variations, the wireless transceiver 130 may be configured to communicate through cellular (e.g., Long Term Evolution (LTE and LTE Cat-M1) networks. Wi-Fi, long range low-power wide area networks (LoRa), Internet of Things (IoT) networks, or any other feasible communication techniques. The first operating mode is described in more detail in conjunction with FIGS. 2 and 3.

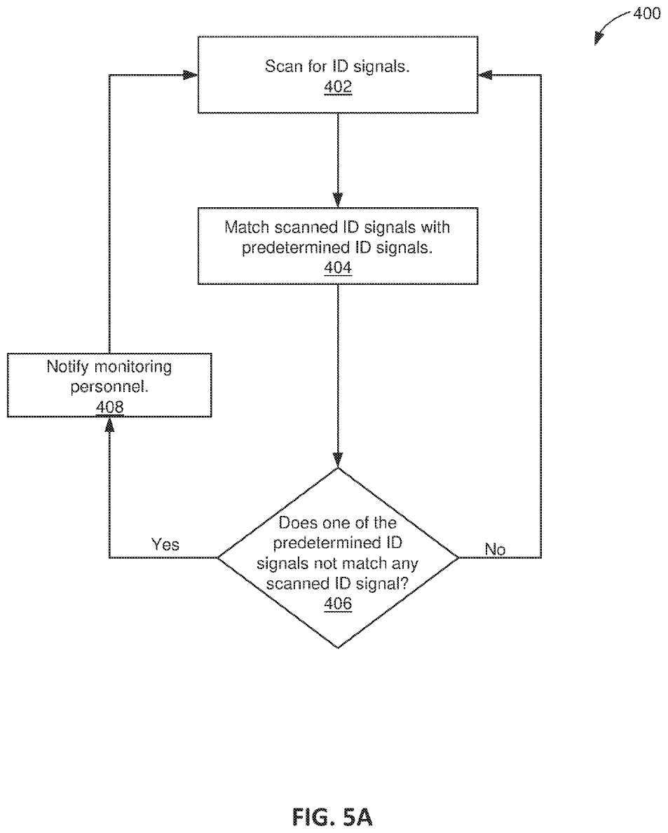

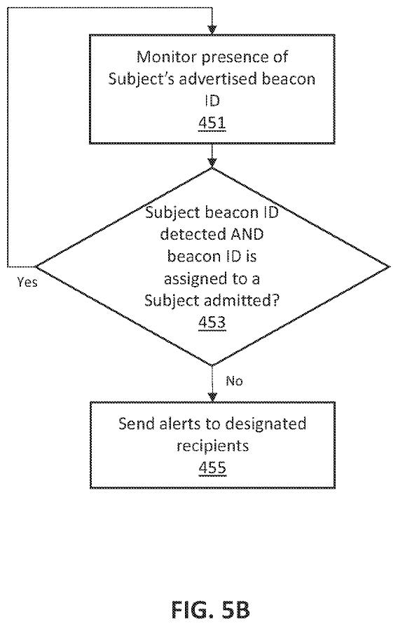

In another operating mode, wireless access points within a defined area or region may scan for a wireless signal transmitted by the wireless transceiver 130. For example, the wireless transceiver 130 may periodically broadcast a unique identification (ID) signal that is associated with the tracking device 100 (and therefore the associated subject). The unique ID signal can be a unique serial number, MAC address or the like associated with the tracking device 100. If the ID signal is not received by the wireless access points, then an alert may be transmitted to monitoring personnel. The second operating mode is described in more detail below in conjunction with FIGS. 2 and 5A.

In some cases, the tracking device 100 may operate in one of the two operating modes. In some other cases, the tracking device 100 may operating simultaneously in both the first and second operating modes. Although only two operating modes are described herein, any number of operating modes are possible. For example, a third operating mode may include one or more aspects of the first and second operating modes. In other words, the third operating mode may be a combination of the first and second operating modes.

FIG. 2A shows a diagram depicting an example deployment of a subject elopement tracking system 200, in accordance with some embodiments. The subject elopement tracking system 200 may include a tracking device 215, one or more access points (shown here as 220(a) and 220(b)), location services 230, backhaul services 240, a monitoring server 250, alert devices 260, and monitoring personnel 270. Although only two access points 220(a) and 220(b) are illustrated here for simplicity, in other variations, any feasible number of access points may be used. Furthermore, although only one monitoring server 250 is shown, the monitoring server 250 may include multiple servers and, in some embodiments, may be distributed across multiple locations to provide redundancy and remote (e.g., cloud) access.

As shown, a subject 210 may wear the tracking device 215. In some variations, the tracking device 215 may be comfortable and tamper-resistant. In some other variations, the tracking device 215 may be an embodiment of the tracking device 100 of FIG. 1. In still other variations, the tracking device 215 may include a display to display time and date, thereby functioning as a watch for the subject 210.

As described above with respect to FIG. 1, the tracking device 215 may operate in at least two different operating modes. In the first operating mode, the tracking device 215 may monitor movements of the subject 210 through included inertial and/or environmental monitoring units. If data from inertial and/or environmental monitoring units exceeds a threshold, then the tracking device 215 may scan for known wireless signals (Wi-Fi beacons, Bluetooth signals or the like) that would indicate that the tracking device 215 is within a predetermined area. For example, Wi-Fi beacons may be transmitted by the wireless access points 220(a) and/or 220(b) within area 225. Wireless signal characteristics (e.g., identifiers, SSID, MAC address, or any other feasible wireless signal characteristics) associated with wireless access points 220(a) and/or 220(b) within area 225 may be stored within a memory of the tracking device 215. If the tracking device 215 is not within the area 225, then the tracking device 215 may not receive any wireless signals associated with the area 225. In some cases, the tracking device 215 may match received wireless signals with the wireless signal characteristics stored within the memory of the tracking device 215. Received wireless signals that do not match stored wireless signal may indicate that the tracking device is not within the area 225. In this manner, the tracking device 215 may determine whether or not the subject 210 is within the area 225. In response, the tracking device 215 may enable location determining operations (for example, through the location determining unit 140) and determine its location. In some variations, the determined location may be reported to the monitoring personnel 270.

For example, the subject 210 may be initially located within an Emergency Room, a hospital bed, or any other feasible location. The location may include one or more wireless access points 220(a) and 220(b). If the tracking device 215 determines that the subject 210 is moving (by determining that data from inertial or environmental sensors exceed a threshold), then the tracking device 215 may scan for known wireless signals. If no known wireless signals are received, then the tracking device 215 can use location services 230 in conjunction with a location determination module in the tracking device 215 (not shown) to determine the location of the subject 210. In some variations, the location determination module can determine a last known location of the subject 210 or a possible path of the subject 210 by recognizing a last known received wireless signal. Such a wireless signals may be provided by a wireless access point positioned at or near a “chokepoint” or doorway of a facility or building.

Operating in the first operating mode may advantageously extend battery life of the tracking device 215. For example, when the subject 210 is confined to a suite or room while they await diagnosis, in-subject transfer or discharge, the subject 210 is relatively stationary. The tracking device 215 may use low-power inertial or environmental monitoring to determine if the subject 210 is moving. If movement is detected, the tracking device 215 can scan for wireless signals. If received wireless signals indicate that the subject 210 has moved to an unknown area (e.g., either no wireless signals are received, or wireless signals are received that do not match wireless signal characteristics stored within the tracking device 215), then the tracking device 215 may enable a location determining module. On the other hand, if no movement is detected (or detected movement is less than a threshold), then no actions may be taken, and battery life can be conserved.

After the tracking device 215 determines its location, the tracking device 215 can send a message to the monitoring server 250. As described with respect to FIG. 1, the tracking device 215 may include a wireless transceiver (not shown). The wireless transceiver may communicate with the monitoring server 250 through any feasible backhaul services 240.

The monitoring server 250 may notify alert devices 260 of the location of the tracking device 215 and subject 210. The alert devices 260 (which may include desktop computers, laptop computers, personal digital assistants, tablet computers, smart phones, or any other feasible device) may receive location information of the subject 210. The alert device 260 may notify the monitoring personnel 270. The alert message may include location information as well as direction of flight of the tracking device 215.

In some embodiments, the monitoring server 250 may determine (compute) the location of the tracking device 215. For example, the tracking device 215 may measure or determine communication information (AoA. AoD, etc.) and transmit the measurement to the monitoring server 250. The monitoring server 250 may have more computational power than the tracking device 215 and may more easily determine the location of the tracking device 215 based on the transmitted measurements.

In the second operating mode, wireless access points (such as the wireless access points 220(a) and 220(b)) within the area 225 may scan for a unique ID signal transmitted by the tracking device 215. In some variations, when an access point receives ID signals, the access point may transmit the ID signals, along with an access point identification information, to the monitoring server 250. In some variations, the access points may communicate with the monitoring server 250 through any feasible backhaul services 240.

The monitoring server 250 can correlate the received ID signals and access point identification information to determine the location of the ID signal. The monitoring server 250 can transmit the location of the tracking device 215 to the alert devices 260.

The methods and apparatuses described herein may be used to extend battery life, in particular by delaying turning on wireless transmitting or receiving or both until the patient is outside of a permitted region (e.g., a room, ward, hall, building, center, campus, facility, etc.). In some cases, even using the receive mode (e.g., wireless receiver) intermittently can shorten the useful battery life of the apparatus. Thus, it may be particularly helpful to minimize or eliminate turning on the wireless receiver (or transceiver) until the apparatus has confirmed that the subject has left the permitted region. As described above, operation of device in a “receive mode” is typically more power intensive than in a transmit mode because the receive “window” has to be open (e.g., “on”) for a period that exceeds the transmit cycle of any home-base beacon. If the home-base transmitter advertises every 60 sec, the receive window of the wearable needs to exceed 60 sec. Conversely, transmission typically takes milliseconds.

In some examples, the patient may remain for a stay length that is relatively long, such as a residential treatment center, which may benefit from especially long periods between requiring recharging or changing of the subject-worn apparatus.

In any of the methods and apparatuses described herein it may be particularly beneficial to further reduce battery consumption associated with even intermittent turning on of a receiver (and in some cases transmitter) in the wearable apparatus, and/or in home-base apparatuses. Thus, although these apparatuses may use the components and techniques (e.g., software, hardware, and/or firmware) described herein to trigger powering on of the energy-hungry locating and backhaul technologies such as GPS or public WiFi access point communication that may be useful when the subject leaves beyond the permitted region. For example, location reporting from the subject worn device may be important (and may be triggered) when the subject goes through a door to the outdoors, or, if outdoors already, when they cross a pre-defined boundary.

In any of these apparatuses and methods described herein a low-frequency (e.g., between about 5-150 kHz, between about 5-140 kHz, between about 5-130 kHz, between about 5-120 kHz, between about 5-110 kHz, between about 5-100 kHz, etc.), and low-power wireless receiver (“sniffer”) may be used to detect the presence of a low-frequency emitter at or around a boundary and/or egress (door, window, etc.). Egress points, which may act as chokepoints for passage by a subject, and/or boundaries into and out of the permitted region may have a low frequency (5-150 kHz) wireless transmitter at the boundary. In practice, the wearable apparatus can be outfitted with a low-power receiver or sniffer to monitor the presence of these low frequency signals. The presence of such low-frequency signals, which may only be present at the chokepoint or boundary region, may indicate that the subject has passed the chokepoint or boundary. If detected, the wearable apparatus (worn by the subject) turn on the location services (e.g., GPS, WiFi access points, etc.) and/or backhaul. Unlike other receivers (e.g., Bluetooth receivers), low-frequency sniffers use very little power. In particular, such apparatuses use much less power than a Bluetooth receiver-sniff. In addition, low frequency transmitters at chokepoint or boundaries may provide a much sharper “roll-off” than Bluetooth. Specifically, the low-frequency transmitters described herein may transmit only over a relatively short distance (e.g., 8 feet or less, 7 feet or less, 6 feet or less, 5 feet or less, etc.) and are not reflected as readily as other frequencies (e.g., Bluetooth). In other words. Bluetooth signals transmit over relatively long distances, and thus, if they are used at a chokepoint or boundary, the wearable apparatus may falsely trigger turning on of the location services (e.g., a false trigger). Due to the sharp roll-off of low frequency signals with low-frequency components, fewer false triggers will occur. This is especially important if the indoor or in-boundary activity takes place close to the boundary, e.g., where the subject may travel past the boundary or choke point without crossing it and leaving the permitted region. In such cases, the use of a low-frequency trigger signal may result in less ambiguity as the patient approaches the boundary or check point, as compared with other signals, such as a Bluetooth signal, that may trigger elopement and turning on of location services.

For example, FIG. 2B is a diagram of an example deployment of a subject elopement tracking system 200′. The subject elopement tracking system may include a tracking device 215′, and a plurality of Bluetooth/Wifi backhaul gateways (e.g., access points) 261, a location services 230, backhaul services 240, and may also include alert devices, and monitoring personnel. Any feasible number of Bluetooth or WiFi (e.g., access points) may be used. As mentioned above, a monitoring server may include multiple servers and, in some embodiments, may be distributed across multiple locations to provide redundancy and remote (e.g., cloud) access. In FIG. 2B, the Bluetooth gateways 261 are configured as Bluetooth-WiFi gateways, which may “sniff” for Bluetooth signals from the subject-worn devices and may relay that information over WiFi to the monitoring server. In some examples, one or more of these Bluetooth gateways may be configured as a home-base Bluetooth beacon that the wearable (subject-worn) device may sniff for to determine whether they are in or out of a permitted area, as described herein.

The permitted region (e.g., emergency room 205) may include a subject observation area (e.g., BH observation area 233). In this example, permitted region is limited to the interior of a building 205 and the patient-worn apparatus may include one or more sensors for detecting a Bluetooth signal indicating that the patient is still within the permitted region. The Bluetooth emitters (or in some cases receivers or transceivers) may be configured to be arranged within the permitted region and may be positions at or near regions of egress (e.g., doors, windows, etc.) as shown in FIG. 2B.

As shown, the subject 210 may wear the tracking device 215′. The tracking device 215 may be comfortable and tamper-resistant. As described above, the tracking device 215′ may operate in at least two different operating modes. In the first operating mode, the tracking device 215′ may monitor movements of the subject 210 within the permitted region (confirming that the patient is still within the permitted region), but the use of one or both of an inertial and/or environmental monitoring units (e.g., Bluetooth data). If data from inertial and/or environmental monitoring units exceeds a threshold, then the tracking device 215′ may scan for activate the location detection sub-system, such as turning on one or more receivers/transceivers to engage with the location services (e.g., wireless beacons 232. GPS 230, etc.) that would indicate that the location of the tracking device 215′. Bluetooth signal characteristics (e.g., identifiers, SSID, MAC address, or any other feasible signal characteristics) may be associated with wireless communication within permitted region and may be stored within a memory of the wearable device 215. If the device 215 is not within the permitted region 205, then the tracking device 215′ may not receive any wireless signals associated with the area 205. Thus, the tracking device 215′ may determine (at a first approximation) if the subject 210 is within the permitted region 205. In response, if outside of the permitted area (based on a received signal or failure to receive a signal) from the Bluetooth gateways 261, the tracking device 215′ may enable location determining operations (for example, through a location determining unit 140, as described above) and by determining its location. In some variations, the determined location may be reported to the monitoring personnel 270.

As discussed above in reference to FIG. 1A, the patient-worn tracking device 215′ may scan for known wireless signals; this may be triggered based on one or more sensors, including light sensors and/or pressure/touch sensors, and/or thermal sensors, humidity sensors, etc. If no known wireless (e.g., Bluetooth) signals are received, then the tracking device 215′ can use location services 230 (e.g., GPS) in conjunction with a location determination module in the tracking device to determine the location of the subject 210 and/or to issues an alert that the patient has left the permitted region.

As mentioned above, the first operating mode may advantageously extend battery life of the tracking device 215′, including using low-power inertial or environmental monitoring to determine if the subject is moving, and if movement is detected, scanning for wireless (e.g., Bluetooth) signals. If received signals indicate that the subject has moved to an unknown area (e.g., either no wireless signals are received, or wireless signals are received that do not match wireless signal characteristics stored within the tracking device 215′), then the tracking device 215 may enable a location determining module. On the other hand, if no movement is detected (or detected movement is less than a threshold), then no actions may be taken, and battery life can be conserved.

The tracking device 215′ can send a message to the monitoring server. As described, the tracking device 215′ may include a wireless transceiver to communicate with the monitoring server through any feasible backhaul services 240. The monitoring server may notify or issue an alert of the location of the tracking device 215′ (and therefore the subject 210).

Any of these method and apparatuses described herein may alternatively or additionally include a low-frequency (e.g., a low-frequency radio loop, such as an AM radio loop, FM radio loop, etc.) emitter at a boundary and/or ingress/egress region (window, door, etc.) as mentioned above. In some examples the wrist-worn apparatus may be configured as mentioned above to include a low-power receiver that detects when the worn apparatus passes over or through the low-frequency emitter, as when a subject passes out of the door or over a boundary to which a wire (e.g., radio loop) is included. In any of these examples, one or more additional sensors (motion sensors, light sensors, temperature sensors, barometric sensors, etc.) may be included in the worn apparatus. When the apparatus confirms that the patient has left the boundary, e.g., of the permitted region, the apparatus may turn on the location services, e. GPS, etc.).

This configuration is illustrated schematically in FIG. 2C, showing a deployment configuration similar to that shown in FIGS. 2A and 2B but with the addition of a low-frequency boundary (e.g., radio look 288). Also in FIG. 2C the wrist-worn apparatus 215″ worn by the patient 210 may wear a wearable apparatus including one or more sensors as well as an emitter for emitting a signal (e.g., Bluetooth). In some examples, as mentioned above, the system may detect a regular transmission by the wearable apparatus while the subject remains within the permitted region 205″ (e.g., interior location(s) and one or more exterior locations). The wrist worn apparatus may also include a low-frequency, low-power sensors for detecting the low-frequency (e.g., less than 150 kHz) signal from the boundary due to the radio loop 234. Upon detecting that the boundary has (likely) been crossed, which may be calibrated by one or more additional sensors as described herein, the wearable apparatus may be switched to activate location serves in the apparatus, as shown in FIG. 2C.

FIGS. 3A-3C illustrate examples of different permitted area layouts that may be used with the apparatuses (e.g., systems) described herein. For example, in FIG. 3A the permitted region is a 45 acre facility that include both internal and exterior permitted regions. In this example the permitted area is fairly large, and therefore a lower frequency signal, e.g., from a radio loop device, may be used. FIG. 3B shows another example of a permitted area layout having a large outdoor area (200 acres) and a number of different buildings. Finally FIG. 3C shows an example in which all of the permitted region is within a particular building.

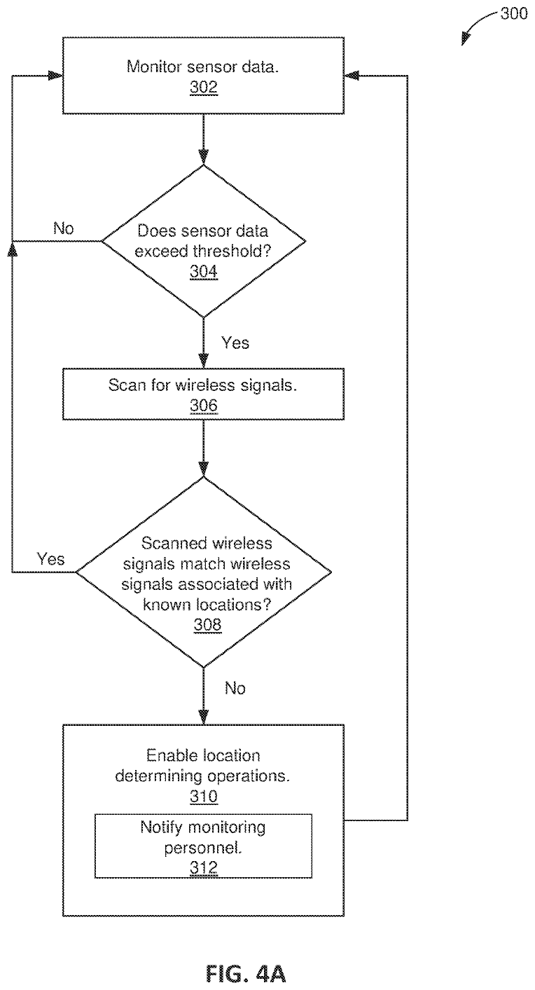

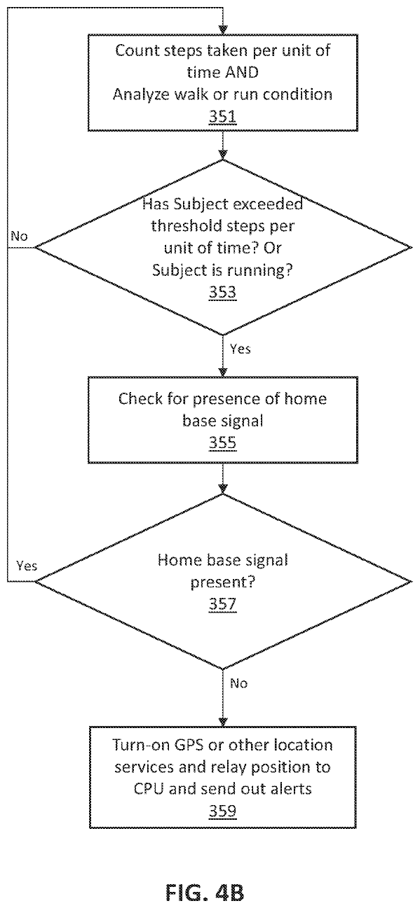

FIG. 4A is a flowchart depicting an example method 300 for tracking a subject, in accordance with some embodiments. Some examples may perform the operations described herein with additional operations, fewer operations, operations in a different order, operations in parallel, and some operations differently. The method 300 is described below with respect to the tracking device 100 of FIG. 1, however, the method 300 may be performed by any other suitable system or device. The method 300 may describe operating the tracking device 215 in the second operating mode described with respect to FIG. 1.

The method 300 begins in block 302 as the tracking device 100 monitors data from inertial and environmental sensors. The inertial sensors may be included within the IMU 110 and sense motion of the tracking device 100. Inertial sensor data may include accelerometer data indicating footfalls (walking, running, and the like) and falls. Environmental sensors may be included in the EMU 120 and sense environmental conditions. Environmental sensor data may include ambient light data and/or ambient temperatures data.

Next, in block 304 the sensor data is compared to a threshold to determine if the threshold has been exceeded. For example, if accelerometer data exceeds a threshold associated with walking (e.g., accelerometer data indicates that the subject is running or has fallen), then the method may proceed to block 306. In a similar manner, if an ambient light sensor detects a change in light (e.g., transitioning from dark to light or light to dark when such a transition is not expected), then the method may proceed to block 306. In another example, if a temperature sensor detects a change in temperature that is greater than a threshold, then the method 300 may proceed to block 306.

In block 306, the tracking device 100 scans for wireless signals. For example, the tracking device 100 may scan for Wi-Fi beacons or other wireless signals that may assist in determining the location of the tracking device 100.

Next, in block 308 the tracking device 100 determines whether any of the scanned wireless signals match wireless signals associated with known locations. For example, the tracking device 100 can compare the received wireless signals (received in block 306) to any known wireless signals. In some variations, the tracking device 100 can compare characteristics of the received wireless signals to characteristics of wireless signals that have been previously stored in a memory of the tracking device 100. The stored wireless signal characteristics may be associated with wireless access points with known or defined locations. If the tracking device 100 matches a received wireless signal with a wireless signal stored in the memory, then the tracking device 100 must be located near a known wireless access point. Thus, the location of the tracking device 100 is known.

If the scanned wireless signals match wireless signals associated with known locations, then the method returns to 302. In other words, since the location of the tracking device 100 is known, the subject is not eloping, and the method can return to 302. On the other hand, if the scanned wireless signals do not match wireless signals associated with known locations, then in block 310 the tracking device 100 may enable location determining services. For example, the tracking device 100 may enable the location determination unit 140 to determine the location of the tracking device 100 using navigation signals (GPS or the like), received wireless communication signals, or through any other feasible method.

In some embodiments, any of a variety of assisted navigation (assisted GPS) techniques may be used to reduce the time necessary for the location determination unit 140 to determine the location (e.g., determine a “fix”) of the tracking device 100. For example, satellite ephemeris information may be pre-downloaded to the location determination unit. Time of day and initial location information (any location information to reduce satellite acquisition time) may also be preloaded to the tracking device 100.

The tracking device 100 may transmit the location through backhaul services (such as backhaul services 240 of FIG. 2A) to the monitoring server 250. In some variations, backhaul services may include open wireless access points, LoRa, LTE, or other feasible wireless communication protocols. Next, in block 312 monitoring personnel are notified. For example, the monitoring server 250 may transmit an alert message indicating a location of the tracking device 100 to monitoring personnel 270. The method may return to block 302.

As described, when operating in the second operating mode, the tracking device 215 simply transmits an ID signal. The simple transmission of the ID signal may require low amounts of power, thereby extending battery life. The ID signal may be transmitted periodically. For example, the ID signal may be transmitted every 600 ms, but other periods are possible. The frequency of transmission may affect the update frequency of the subject's location. The frequency of transmission may also affect battery life of the tracking device 215.

In some embodiments, a geofence area may be defined. The geofence area may be an area that may be within a monitored area, but just beyond wireless signal (e.g., Wi-Fi signal) coverage. Thus, the geofence area may be within a controlled area where the subject may travel without eloping. When the tracking device 215 is within the geofence area, the location of the tracking device 215 may be reported less frequently to the monitoring server 250 to inform monitoring personnel of the subject's location without depleting the battery.

FIG. 4B illustrates another example of a method of determining that the patient has eloped and/or tracking the patient. In FIG. 4B, the subject is wearing the tracking device. The tracking device may detect subject locomotion data (e.g., walking, running, etc.) using an accelerometer 351. The accelerometer may, for example, count steps taken per unit time and analyze the rate to determine if the subject is walking or running. In some examples, an estimate for distance traveled may be made (e.g., based on the duration, rate of travel/number of steps and changes in direction, all of which may be determined from the IMU data), and compared to a distance threshold. Thus, the controller in the device may compare the sensed data (e.g. subject locomotion data) to one or more thresholds 355.

During this time, the device may operate in a low-power mode, in which it is monitoring the data (e.g., IMU/EMU data) and optionally in some examples, may be transmitting (a lower-power operation than receiving). If the controller determines that the one or more thresholds have been exceeded, indicating elopement is likely, the controller may then activate the wireless transceiver to check for the presence of a wireless signal characteristic of the “home base” condition 355. For example, the apparatus may determine if the wireless network is within range (or within range with a set power threshold). If the controller determines that the home base signal is not detected (or not detected within the appropriate power range), the controller may then activate (enable) the location-determining operations in the tracking device to detect position. e.g., turning on a GPS service 359. A GPS chip may be activated and may be used to give location information with a relatively high frequency (e.g., faster than once every five minutes, once every four minutes, once every three minutes, once every two minute, once every minute, once every 45 seconds, once every 30 seconds, etc.). The device may report the location information to a remote server, e.g., using backhaul technology (e.g., cellular, IOT LT-M or CAT-M1, or any other long-range IOT radio, such as LORA).

FIG. 5A is a flowchart depicting another example method 400 for tracking a subject. The method 400 is described below with respect to the subject elopement tracking system 200 of FIG. 2A, however, the method 400 may be performed by any other suitable system or device.

The method begins in block 402 as access points 220(a) and 220(b) scan for ID signals transmitted by the tracking device 215. The ID signals may be unique to each tracking device 215 and, therefore, unique to each subject 210 wearing or possessing the tracking device 215. In some embodiments, the tracking device 215 may transmit the ID signals periodically. For example, the tracking device 215 may transmit the ID signal every 600 ms, however other periods are possible. Such a period may provide location updates that are sufficiently fast to track a moving subject.

Next, in block 404 the monitoring server 250 matches scanned ID signals with predetermined ID signals. Predetermined ID signals may be ID signals associated with subjects that are expected to be within area 225. In other words, the monitoring server 250 can determine whether the subjects that should be in area 225 (the subjects in a list of the predetermined ID signal) are physically in the area 225 (the subjects associated with the scanned ID signals). In some variations, the predetermined ID signals may be stored within a memory of the monitoring server 250. In some other variations, the predetermined ID signals may be stored in a memory coupled to and/or remotely accessible by the monitoring server 250.

Next, in block 406, the monitoring server 250 determines whether at least one of the predetermined ID signals do not match any of the scanned ID signals. If a predetermined ID signal does not match any of the scanned signals, then the subject 210 associated with that predetermined ID signal is missing from the area 225. Thus, the method proceeds to block 408 where monitoring personnel are notified. This notification may be similar to notifying monitoring personal discussed above with respect to block 312. The method then returns to block 402.

Returning to block 406, if all of the predetermined ID signals can be matched with a scanned ID signal, then all subjects 210 associated with area 225 are present. No notification messages need to be sent and the method returns to 402.

Notably, the periodic or rapid scanning (performed in block 402) may be performed by devices that are powered by AC power and not subject to limited power, such as battery power. Advantageously, the tracking device may simply transmit a low-power ID signal, thereby conserving power and extending battery life.

FIG. 5B illustrate another example of a method for tracking a subject similar to that described in FIG. 5A. In FIG. 5B the method may include monitoring the presence of a subject's advertised beacon ID 451, determining if the subject's beacon ID is detected and if the beacon ID is assigned to a subject that has been admitted to the monitoring group 453. If so, the system may continue to monitor the presence of the subject's beacon. If not, the system may send an alert to designated recipient's 455.

Methods (and apparatuses for performing them) such as those shown in FIGS. 5A-5B may work with multiple patients, rather than just one or a few (e.g., more than 2 patients, more than 3 patients, more than 4 patients, more than 5 patients, more than 10 patients, more than 15 patients, more than 20 patients, more than 25 patients, more than 50 patients, etc.). The gateway receiver may be continuously or discretely/periodically (e.g., constantly) listening for the beacons and there may be a large number of patients (e.g., in one example, 20 or more patients). The system may look for the presence of each patient, then looking to see if beacon is assigned to patient. Devices that are not used (and or stored or charging, or in standby) may still beacon, but the database will show not assigned. If a missing beacon signal is assigned to a non-discharged subject, then the system may send out an alert of elopement.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A method for monitoring and tracking a subject that preserves battery life in a tracking device worn by a subject, the method comprising:
monitoring sensor data in the tracking device;
determining whether the sensor data exceeds a threshold;
turning on scanning for a wireless signal when the sensor data exceeds the threshold; and
enabling location-determining operations when the scanned wireless signal does not match a wireless signal associated with a known location.

2. The method of claim 1, wherein the sensor data is subject movement sensor data, environmental sensor data, or a combination thereof.

3. The method of claim 2, wherein the subject movement sensor data is accelerometer data, footfall data, falling data, or a combination thereof.

4. The method of claim 2, wherein the environmental sensor data is ambient temperature, ambient light data, or a combination thereof.

5. The method of claim 1, wherein the wireless signal is a signal transmitted by a wireless access point.

6. The method of claim 5, wherein the wireless signals is a Wi-Fi beacon signal.

7. The method of claim 1, wherein the wireless signals is a Wi-Fi signals, a Bluetooth signals, or a combination thereof.

8. The method of claim 1, wherein the wireless signals associated with the known location is stored in a memory of the tracking device.

9. The method of claim 1 further comprising: determining a location of the tracking device based on the location determining operations; and notifying monitoring personnel in response to determining the location of the tracking device.

10. The method of claim 9, wherein notifying monitoring personnel includes transmitting a determined location of the tracking device to the monitoring personnel.

11. The method of claim 1, wherein the location determining operations are performed every minute or more frequently.

12. The method of claim 1, wherein enabling location-determining operations comprises determining using global positioning system (GPS) signals indicating the location of the tracking device.

13. The method of claim 1, wherein enabling location-determining operations comprises using one or more of: global positioning system (GPS) signals indicating the location of the tracking device, public WiFi signals, barometric pressure.

14. The method of claim 1, further comprising turning off scanning for the wireless signal if the wireless signal matches the wireless signal associated with the known location.

15. A method for tracking a subject that preserves battery life of a tracking device worn by the subject, the method comprising:
monitoring subject locomotion using the tracking device, wherein the tracking device includes one or more motion sensors;
determining whether the subject locomotion exceeds a distance threshold and/or exceeds a rate threshold indicating running;
turning on scanning for a wireless signal by the tracking device if the subject locomotion exceeds the distance threshold and/or exceeds the rate threshold indicating running; and
enabling location-determining operations in the tracking device if the tracking device identifies a wireless signal that matches a known location, wherein the location-determining operations include turning on location services and relaying position from the tracking device to a remote processor at a rate of 0.02 Hz or greater.

16-25. (canceled)

26. A method for tracking a subject, the method comprising:
scanning, by one or more wireless access points, for identification signals associated with individual subjects;
matching the scanned identification signals with predetermined identification signals, wherein the predetermined identification signals are associated with individuals expected to be within wireless range of the one or more wireless access points;
determining that at least one of the predetermined identification signals fails to match with the scanned identification signals; and
notifying monitoring personnel in response to determining that at least one of predetermined identification signals fails to match with the scanned identification signals.

27. The method of claim 26, wherein the identification signals are transmitted by a wireless transmitter worn by individual subjects.

28. The method of claim 26, wherein the identification signals include Wi-Fi signals, Bluetooth signals, Zigbee signals, or a combination thereof.

29. The method of claim 26, wherein the one or more wireless access points are coupled to a monitoring server configured to match scanned identification signals to the predetermined identification signals.

30. The method of claim 26, wherein the notifying includes transmitting alert messages to the monitoring personnel.

31. The method of claim 30, wherein the alert message is transmitted to at least one of a desktop computer, laptop computer, personal digital assistant, tablet computer, and smart phone.

32. The method of claim 30, wherein the alert message is transmitted by a server coupled to the one or more wireless access points.

33-37. (canceled)
